(19) United States
(12) Patent Application Publication
(10) Pub. No.: US 2026/0144440 A1
(43) Pub. Date: May 28, 2026

(54) AUGMENTED VISION EXAMINATION TECHNIQUES USING MACHINE LEARNING

(71) Applicant: DigitalOptometrics LLC, Lake Success, NY (US)

(72) Inventors: William Kenneth Van Cleave, Abilene, TX (US); Kurt Schaeffer, Lake Success, NY (US); Gordon Durgha, Lake Success, NY (US); Robert Hansel, Lake Success, NY (US); Paul E. Muehlhausen, Lake Success, NY (US); Mobin Varghese, Lake Success, NY (US); Charles A. Dowalo, Lake Success, NY (US); Howard S. Fried, Lake Success, NY (US); Alex Louw, Lake Success, NY (US); Douglas C. Viney, Lake Success, NY (US)

(21) Appl. No.: 19/398,465

(22) Filed: Nov. 24, 2025

Related U.S. Application Data
(60) Provisional application No. 63/724,538, filed on Nov. 25, 2024.

(51) Int. Cl. A61B 3/028; A61B 3/00; G10L 15/02; G10L 15/22; G10L 25/90; G16H 10/60

(52) U.S. Cl. CPC A61B 3/0285; A61B 3/0025; A61B 3/0041; A61B 3/0075; G10L 15/02; G10L 15/22; G10L 25/90; G16H 10/60; G10L 2015/025; G10L 2015/223

(57) ABSTRACT

Systems and techniques are disclosed for augmenting and/or automating aspects of vision examinations. In some implementations, context data is obtained from a plurality of data sources. A candidate command for adjusting a hardware component of optometric equipment is determined based on the current state of the vision examination. Prompt data for one or more trained machine learning models is generated based on the candidate command. The prompt data is provided to a hosting system associated with the one or more trained machine learning models. Model output data generated by the one or more trained machine learning models is obtained from the hosting system in response to providing the prompt data. The model output data is parsed based on the candidate command to generate one or more command-specific executable instructions for the hardware component. Output data representing the one or more command-specific executable instructions is provided for output.

### CROSS-REFERENCE TO RELATED APPLICATIONS

This application claims the benefit of U.S. Provisional Application No. 63/724,538, filed on Nov. 25, 2024, the contents of which are incorporated by reference in its entirety.

### TECHNICAL FIELD

This disclosure generally describes technology relating to machine learning, and more particularly, to integration of machine learning into tele-optometry systems.

### BACKGROUND

Machine learning (ML) enables systems to learn from data and improve their performance without being explicitly programmed for every task. Rather than following predefined rules, ML systems build models based on patterns found in large datasets. These models can then make predictions, classify data, or perform decision-making tasks based on new, unseen data. ML may involve providing input data to a trained model, which processes the provided data to identify patterns or relationships within the data.

ML may involve several types of learning. For example, in supervised learning, a model is trained on labeled data, where both the inputs and desired outputs are known. The goal is to learn a mapping from inputs to outputs to make predictions on new, unlabeled data. As another example, in unsupervised learning, a model works with data that has no labeled outcomes. As another example, in reinforcement learning, a model learns by interacting with an environment and receiving feedback in the form of rewards or penalties. ML has applications across industries, including healthcare, finance, and consumer technologies. In the context of healthcare, ML systems and techniques may be useful to predict diseases, analyze medical images, and provide other advantages.

For example, the systems and methods disclosed herein can accommodate technicians of varying skill levels. Using the ML techniques disclosed herein, a technician with minimal training can conduct a vision examination on a variety of ophthalmic equipment, such as various phoropters. One or more agentic models implemented on the system receive and analyze verbal responses from a patient and output data. The output from the system, generated by the agentic models, can be in the form of questions or instructions for the patient, or instructions for the phoropter. In one implementation, the instructions are automatically implemented by the phoropter. In a different implementation, the instructions can be implemented by the technician on the phoropter. As such, the ML models augment the vision examination by interacting with the patient and generating output instructions for the phoropter. While technicians with lower skill levels for a given phoropter may rely more heavily on the instructions output from the system to conduct the entire vision examination, technicians with higher skill levels may use the output to confirm or verify specific aspects of the vision examination.

Examples of eye-focused healthcare industries include optometry, ophthalmology, tele-optometry, and optical retail and services. These industries involve a vision examination (e.g., eye exam), which is a comprehensive evaluation of a person's eyesight and overall eye health. A vision examination is typically conducted by an optometrist, ophthalmologist, or refractionist. The vision examination may involve a series of tests to assess visual acuity (sharpness of vision), determine the need for corrective lenses (such as glasses or contact lenses), and check for common eye conditions such as astigmatism, nearsightedness (myopia), farsightedness (hyperopia), or presbyopia.

A vision examination may include assessing eye movement, coordination, depth perception, peripheral vision, and the health of the internal and external structures of the eye (e.g., retina, cornea, and optic nerve). Vision examinations are conducted using optometric equipment, such as a phoropter, autorefractor, or retinoscope, which measure refractive errors and establish corrective prescriptions. Dilation or imaging techniques may also be employed to examine the health of the eye in more detail. Vision examinations may involve a subjective refraction, which is a technique to determine the combination of lenses that will provide the best corrected visual acuity (BCVA). A subjective refraction examination is a clinical procedure used by orthoptists, optometrists, and ophthalmologists to determine a user's need for refractive correction in the form of glasses or contact lenses.

### SUMMARY

This disclosure describes systems and techniques for augmenting and/or automating aspects of vision examinations administered in a remote or distributed environment. In various implementations, a central server orchestrates the examination by communicating over a network with a client device at an examination site and an external hosting system associated with one or more ML models. The server is configured to receive and analyze context data from multiple sources, including historical patient data, real-time input from the patient, and commands from remote human operators, such as technicians or providers.

Examples of system operation involve a multi-stage process where the server determines a candidate command for adjusting a hardware component of optometric equipment. Based on this command, the server generates prompt data to query one or more ML models for a corresponding parameter or validation. The server parses the resulting model output data to generate one or more command-specific executable instructions that are provided to the optometric equipment to cause a change in its configuration. This architecture enables a variety of examination formats, from human-in-the-loop augmentation to full automation, thereby improving the efficiency, accuracy, and accessibility of remote vision care.

The systems and methods described herein provide technical solutions to data privacy issues implicated in application of large-scale ML models to remote healthcare diagnostics, and specifically to the real-time administration of vision examinations. These solutions involve context-preserving anonymization, which improves the functioning of computing systems by enabling the safe and effective application of ML models in a regulated healthcare environment. As discussed herein, a server is configured to act as a trusted intermediary or “privacy guard” and thereby establishes a secure boundary between sensitive patient data and an external hosting system that makes ML models accessible for use.

Further, conflict may arise from operational requirements of ML systems when applied in healthcare environments. For instance, for an ML model to generate high-quality, clinically relevant, and non-hallucinated outputs, the ML data typically requires prompt data with rich contextual information. This information includes not only a patient's immediate responses, but also includes historical information (e.g., medical history, demographic information), and time-dependent state data (e.g., state of the ongoing examination). However, such information also may represent Personally Identifiable Information (PII) or Protected Health Information (PHI), which is subject to strict data privacy and security regulations, such as the Health Insurance Portability and Accountability Act (HIPAA). Regulations limit the transmission of PII and PHI to external, third-party systems, such as hosting systems providing access to ML models.

This data-privacy paradox creates a significant technical barrier to the effective use of powerful, general-purpose ML models in this field. Conventional approaches to data security are often insufficient and create a technical trade-off that results in system failure. A naive “over-anonymization” approach, which involves stripping all potentially identifying information from the prompt data, renders context data barren and leads to the ML model producing generic, inaccurate, or clinically useless outputs, thereby defeating the purpose of its use. Conversely, an “under-anonymization” approach that transmits the necessary context data without modification would violate privacy laws and create unacceptable security risks, making such a system technically infeasible for deployment in any real-world clinical setting. Therefore, specific, unmet needs exist for computer-implemented systems that can resolve this conflict by intelligently and precisely anonymizing context data in a manner that preserves the contextual integrity required for high-fidelity ML model performance while ensuring strict compliance with data privacy standards.

The systems and techniques described herein address these and other unmet needs through use of a centralized server configured to receive and analyze raw context data containing PII/PHI within its trusted environment. The server performs a specific, context-preserving anonymization process to transform this raw data into a de-identified (yet still contextually rich) format. The server constructs prompt data that contains only this transformed, anonymized data for transmission to an external hosting system. This process represents a specific improvement to computer security and data processing, as it allows the system to leverage the analytical power of large-scale (and, some instances, general-purpose) ML models without exposing sensitive data, thereby overcoming a fundamental technical barrier in the field of remote medical diagnostics.

The systems and methods described herein also provide technical solutions to challenges of network latency and jitter that are inherent in administering real-time, interactive medical examinations over a network. These solutions involve a specific server-centric architecture that manages the feedback loop between a remote patient and the optometric equipment. This architecture is configured to reduce the number of required network round-trips and make the examination process more resilient to network-induced delays, thereby improving the accuracy of the final prescription.

Further, significant technical problems may arise when conducting a vision examination that relies on a patient's immediate perception, over a standard computer network. The process is a time-sensitive, “call-and-response” feedback loop between the patient's subjective state and the machine's physical configuration. Network impairments such as latency, jitter, and packet loss degrade this process, causing a noticeable delay between a remote operator's command, the equipment's adjustment, and the patient's perception of the change. This delay can confuse the patient, making it difficult for them to accurately recall and compare successive options (e.g., “option one” versus “option two”), which may lead to unreliable feedback and a suboptimal clinical outcome.

The systems and methods that address the latency issues discussed above thereby represent specific improvements to computer-related technology, and particularly to the operation of a computer as a remote control system for medical devices. As described herein, the systems are configured to be intelligent and stateful such that they improve functioning by locally determining the next logical clinical step, using targeted and compact data exchanges with the ML model, and then executing the final instruction. This specific architecture transforms a fragile, high-latency, and error-prone manual process into a robust, efficient, and reliable automated process, thereby overcoming a fundamental technical barrier in the field of tele-optometry.

Techniques are also described to improve the efficiency of a networked computer system by implementing a specific data transformation and bandwidth reduction technique. This involves a server configured to process large, raw context data streams locally and transform them into small, information-dense prompt data packets for an external machine learning model. This reduces the network resources required to administer the remote vision examination.

Conventional approaches to remote medical diagnostics often rely on streaming high-bandwidth data, such as continuous, high-resolution video and audio feeds, from the examination site to a remote human operator. This approach is technically inefficient, consuming significant network bandwidth and processing power on both the client and server systems. Reliance on high-bandwidth streams creates a technical bottleneck that limits the scalability of remote examinations, increases operational costs, and makes the system vulnerable to failure on connections with limited or unstable network capacity.

The systems and methods described herein provide a technical solution that enables a computer system to perform a real-time, multi-factor clinical data synthesis that is beyond the practical capabilities of a human operator. The system is configured to analyze and synthesize multiple, disparate data streams simultaneously, including a patient's immediate verbal response, their complete historical clinical data, their demographic profile, and even visual cues derived from video analysis, to inform the examination process.

Further, a human operator, whether remote or local, is subject to inherent cognitive limitations that create a technical barrier to the quality of a manually conducted examination. A human cannot, in the sub-second timeframe required for a smooth and efficient interactive test, simultaneously process a patient's subjective verbal feedback while also correlating it with a specific note from their medical record three years prior, their statistical likelihood to have a certain condition based on their age, and subtle visual indicators of eye strain visible on a video feed. This limitation means that manually conducted examinations are often based on an incomplete set of the available data.

This process represents a specific improvement to computer-related technology because it enables the computer to perform a new and more powerful function that could not be practically achieved by a human. The system improves the computer by transforming it from a simple data relay and execution device into a powerful diagnostic synthesis engine. By performing this multi-factor analysis in real-time to determine a candidate command or generate a model output, the invention enables the computer to facilitate a more accurate, more clinically insightful, and more efficient vision examination than was previously possible.

The systems and methods described herein provide a technical solution for improving the reliability and safety of ML-driven medical systems by implementing a specific and unconventional technical architecture for the use of the ML model. This solution involves integrating the machine learning model as a specialized component within a larger, deterministic control loop managed by the server, rather than employing the model as a monolithic, end-to-end decision-maker.

Further, the use of general-purpose, “black box” machine learning systems in medical applications presents significant technical problems related to reliability, transparency, and safety. Conventional end-to-end ML systems that take all raw data as input and produce a final clinical output are often brittle, difficult to validate, and inexplicable. If such a system produces an erroneous or clinically inappropriate output, it can be difficult to identify the source of the error, to override the decision, or to ensure the system operates within safe clinical guardrails. This lack of transparency and reliability is a major technical barrier to the adoption of such systems.

This specific technical architecture represents an improvement to computer-related technology because it makes the application of machine learning in a clinical context more robust, fault-tolerant, and efficient. The server's function of first determining a candidate command provides a deterministic, rule-based, and clinically safe guardrail for the system's operation. The machine learning model is used only for a well-defined and constrained sub-task, such as interpreting a specific response or estimating a single parameter value. This improves the computer system's overall reliability, safety, and explainability, making it technically suitable for deployment in a real-world clinical environment.

The systems and methods described herein also enable a spectrum of examination formats (e.g., human-in-the-loop augmentation, full automation) and the specific level of automation can be dynamically adjusted based on the examination format. For example, the server may be configured to augment or automate activities based on the patient's demographic profile or their performance during a prior vision examination. If a patient is elderly and has a history of significant vision impairment, the server may be configured to reduce its reliance on fully autonomous processes, ensuring more human oversight. Conversely, for a younger patient with excellent vision health, the server may increase its reliance on ML models to perform a more fully automated examination, thereby increasing efficiency.

In addition to the advantages described above, the system supports multiple languages, enabling global collaboration. The system can use context data, including high-quality, previously generated patient data to provide guidelines for generating inputs to the one or more ML models. The system provides scalability and flexibility for multiple applications with scalable compute and storage resources to adapt to an increasing volume of data. Similarly, the system can be adapted to support multiple phases of a vision examination. The system leverages the feedback loop and adaptive learning to tailor prompts to generate improved vision examinations.

In one general aspect, a method may be implemented by one or more computing devices. The method includes obtaining context data from a plurality of external data sources. The context data includes (i) historical vision examination data for a patient received from one or more databases, (ii) a current state of a vision examination being administered for the patient. The method also includes determining a candidate command for adjusting a hardware component of optometric equipment based on the current state of the vision examination and generating prompt data for one or more trained ML models based on the candidate command. The prompt data specifies a natural language query of the candidate command for a proposed change to a configuration of the hardware component. Further, the method includes providing the prompt data to a hosting system associated with the one or more trained ML models, obtaining model output data generated by the one or more trained ML models from the hosting system in response to providing the prompt data, and parsing the model output data based on the candidate command to generate one or more command-specific executable instructions for the hardware component. The method also includes providing output data representing the one or more command-specific executable instructions for output. When received by a client device, the output data causes the client device to initiate a change to a configuration of the hardware component.

One or more implementations may include the following optional features. For example, in some implementations, the context data further includes input data from the patient received from a remote patient device during the administration of the vision examination. In such implementations, the candidate command is determined based on the historical vision examination data and the input data from the patient.

In some implementations, generating the prompt data includes structuring the query to integrate the candidate command, the historical vision examination data, the current state of the vision examination, and the input data from the patient.

In some implementations, the natural language query is configured to elicit a value corresponding to the candidate command. Further, parsing the model output data includes extracting the value. In such implementations, the one or more command-specific executable instructions are generated based on the candidate command and the value.

In some implementations, the method further includes receiving, from the remote patient device, data indicative of speech from the patient. In such implementations, method also includes converting the data indicative of speech to data indicative of text, where the input data from the patient comprises the data indicative of text.

In some implementations, the method further includes generating an audio response for the patient. In such implementations, the audio response includes a verbal prompt corresponding to the candidate command. Further, the method includes providing the audio response for output to the remote patient device.

In some implementations, the method further includes receiving, from the remote patient device, data indicative of speech from the patient. In such implementations, generating the audio response includes determining one or more waveforms from the data indicative of speech, and generating one or more response waveforms based on the determined waveforms, wherein the audio response comprises the one or more response waveforms.

In some implementations, the method further includes receiving, from the remote patient device, data indicative of speech from the patient. In such implementations, generating the audio response includes determining a pitch and a rhythm of the data indicative of speech. The method further includes generating one or more response waveforms based on the determined pitch and rhythm. The audio response also includes the one or more response waveforms.

In some implementations, generating the one or more response waveforms further includes obtaining, from the hosting system, data indicative of phonemes corresponding to the determined pitch and rhythm and applying the data indicative of phonemes to modulate the one or more response waveforms.

In some implementations, the optometric equipment includes a phoropter. In such implementations, the candidate command is a command to perform an adjustment selected from a group of techniques. The group includes adjusting a spherical lens power, adjusting a cylindrical lens power, adjusting an axis of a cylindrical lens, adjusting a position of a Jackson Cross Cylinder (JCC) lens, adjusting an add power, and adjusting an occluded state of a lens.

In some implementations, the client device includes a phoropter control device. In such implementations, the client device, each of the one or more command-specific executable instructions specifies (i) a command identifier for the adjustment and (ii) a parameter that quantifies a degree of the adjustment. In such implementations, the output data is formatted for an Application Programming Interface (API) of the phoropter control device. Additionally, receipt of the output data by the phoropter control device causes execution of the one or more command-specific executable instructions to perform the adjustment.

In some implementations, the change to the configuration includes updating an eye chart displayed on a graphical user interface of the client device.

In some implementations, parsing the model output data includes determining a degree of similarity between the model output data and one or more stored responses. Additionally, in response to determining that the degree of similarity satisfies a similarity threshold, the method includes generating the one or more command-specific executable instructions. Further, in response to determining that the degree of similarity does not satisfy the similarity threshold, the method includes generating an output inquiry.

In some implementations, the method includes incrementing a counter in response to generating the output inquiry. Further, in response to determining that the counter satisfies a count threshold, the method includes establishing a network connection between the client device and a remote provider device.

The details of one or more implementations of the disclosure are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram of an exemplary system for providing ML-assisted vision examinations.

FIGS. 2A-2E are conceptual diagrams of examination formats for different ML-assisted vision examinations.

FIG. 3 is a conceptual diagram of an exemplary interface for facilitating ML-assisted vision examinations according to the examination formats shown in FIGS. 2A-2E.

FIG. 4 is a block diagram of exemplary communication flows involved in ML-assisted vision examinations according to the examination formats shown in FIGS. 2A-2E.

FIG. 5 is a block diagram of an exemplary logical architecture for processing data associated with ML-assisted vision examinations according to the examination formats shown in FIGS. 2A-2E.

FIG. 6 is a flow chart illustrating an exemplary process of enabling ML-assisted vision examinations.

In the drawings, like reference numbers represent corresponding parts throughout.

### DETAILED DESCRIPTION

This disclosure describes systems and methods for augmenting or automating aspects of a vision examination administered in a remote or distributed environment. In general, the systems utilize a central server to orchestrate the examination process by communicating over a network with a client device at an examination site and an external hosting system associated with one or more ML models. The server is configured to receive and analyze multifaceted context data from a plurality of sources, including historical patient data, real-time patient input, and commands from human operators.

As discussed below, system operation involves a multi-stage process where the server determines a candidate command for adjusting optometric equipment, generates specific prompt data to query one or more ML models for a corresponding parameter or validation, parses the resulting model output data, and generates one or more command-specific executable instructions to effect a change in the equipment's configuration. This flexible architecture enables a variety of examination formats with different levels of ML-assisted automation. In some examples, the system enables human-in-the-loop augmentation, where ML models provide decision support to a human operator. In other examples, the system enables full automation, where ML models autonomously conduct the examination. These examples improve the efficiency, accuracy, and accessibility of remote vision care.

As described herein, “machine learning” refers to a class of computational techniques and models, including to neural networks, transformer-based architectures, generative artificial intelligence, decision trees, support vector machines, clustering algorithms, and statistical learning methods. These techniques and models enable a computer system to automatically learn patterns or representations from data and improve performance on a given task without being explicitly programmed with task-specific rules. Machine learning systems may operate in supervised, unsupervised, semi-supervised, reinforcement, or self-supervised learning paradigms, and may be designed to perform a wide range of tasks such as classification, prediction, generation, translation, anomaly detection, and optimization across various data modalities, including text, images, audio, video, and structured data.

As described herein, a “model” refers to a computational system, algorithm, or structured representation used with a machine learning system. Examples of models include ML models, neural networks, transformer-based architectures, generative models, reasoning models, agentic systems, probabilistic models, statistical models, or rule-based systems. Models may be designed to process input data and produce outputs, predictions, decisions, actions, representations, or generated content. Models may operate under various learning paradigms, including supervised, unsupervised, semi-supervised, reinforcement, or self-supervised learning, and may be configured to perform tasks such as classification, regression, recommendation, anomaly detection, generation, translation, summarization, planning, decision-making, or multi-step reasoning across a range of data modalities, including structured data, text, images, audio, video, and sensor data.

As described herein, a “module” generally refers to a discrete, encapsulated software unit that implements a defined subset of functionality within a larger system. For example, a module may include executable code, data structures, and associated interfaces that collectively enable the module to perform one or more tasks, operations, or services. In some implementations, a module may expose an API or inter-process communication interfaces through which other system components (e.g., agents, tools, or orchestration engines) may invoke module functionality. The module may be configured for local execution within an application runtime or for remote execution via a distributed service environment.

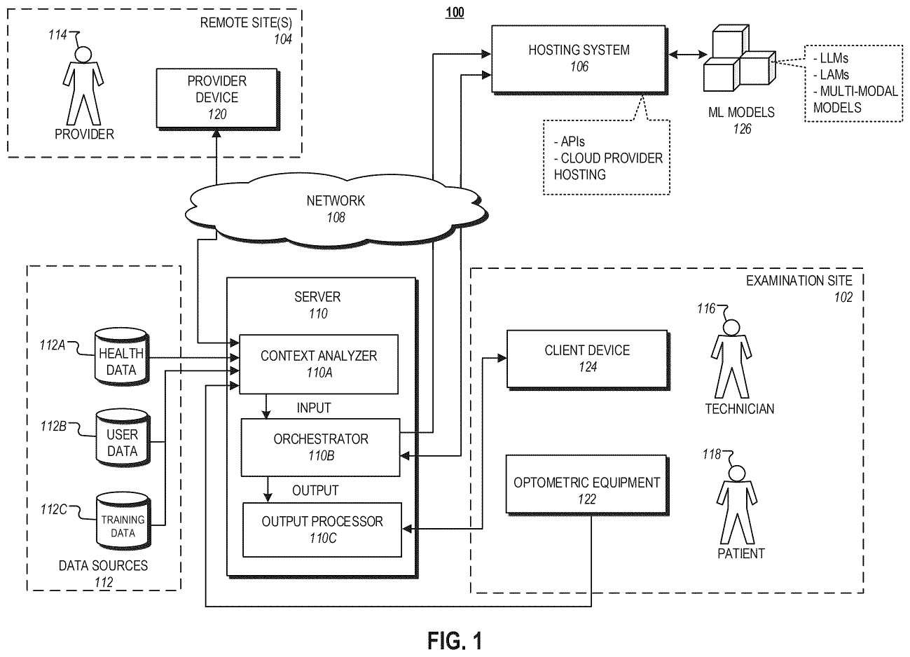

FIG. 1 is a block diagram of an exemplary system 100 for enabling ML-assisted vision examinations. The system 100 includes an examination site 102, which includes optometric equipment 122 and a client device 124 used by a patient 118 and technician 116. The system 100 also includes one or more remote site(s) 104, which include a provider device 120 used by a provider 114, a server 110 that accesses a plurality of data sources 112, and a hosting system 106 that provides access to one or more ML models 126. As shown in FIG. 1, the computing elements of system 100 may be communicatively coupled via a network 108.

The examination site 102 represents a physical location where the vision examination is administered. The examination site 102 includes the optometric equipment 122 (e.g., a phoropter) and a client device 124 that the patient 118 and/or a local technician 116 may use to interact with the system 100. The remote site(s) 104 represent one or more locations that are physically separate from the examination site 102. A remote provider 114 uses a provider device 120 at a remote site 104 to administer or supervise the vision examination over the network 108.

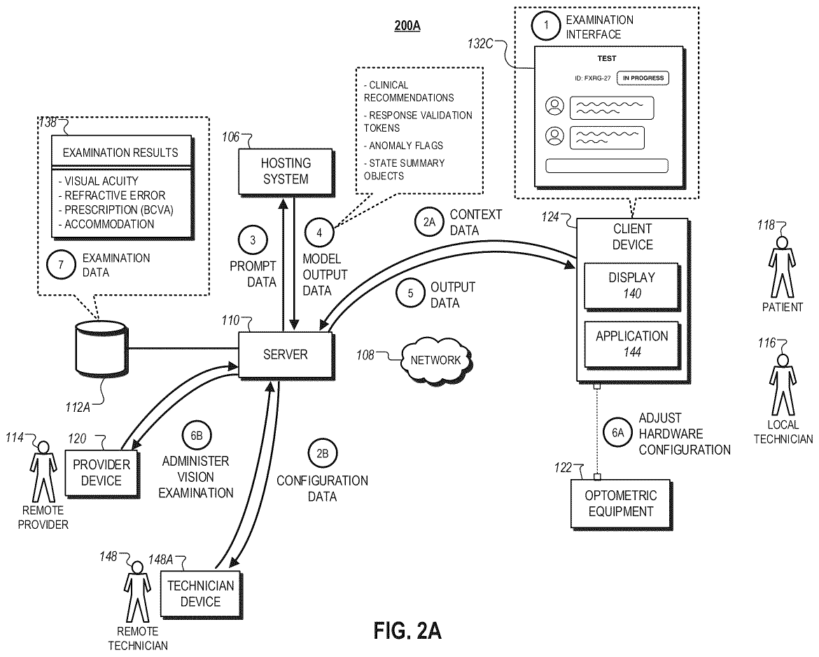

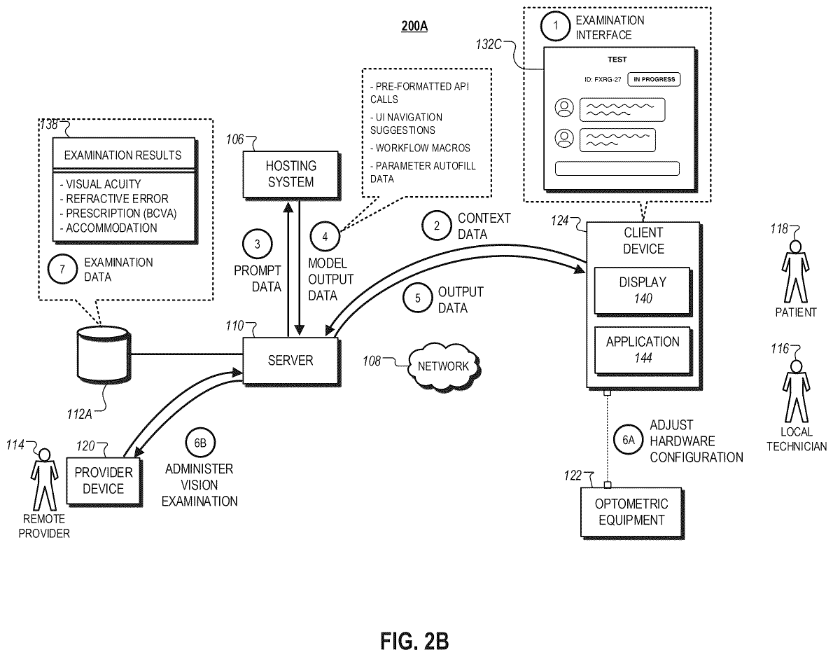

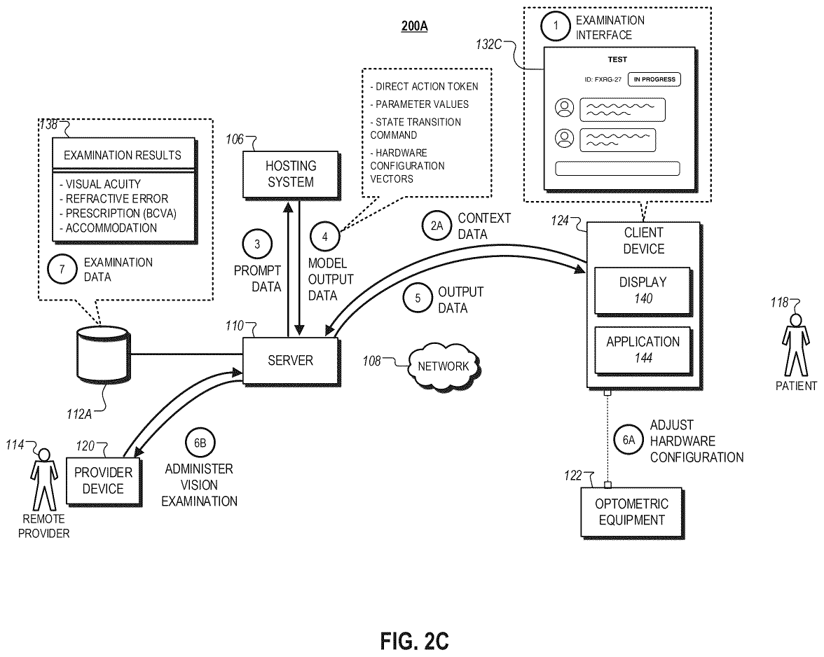

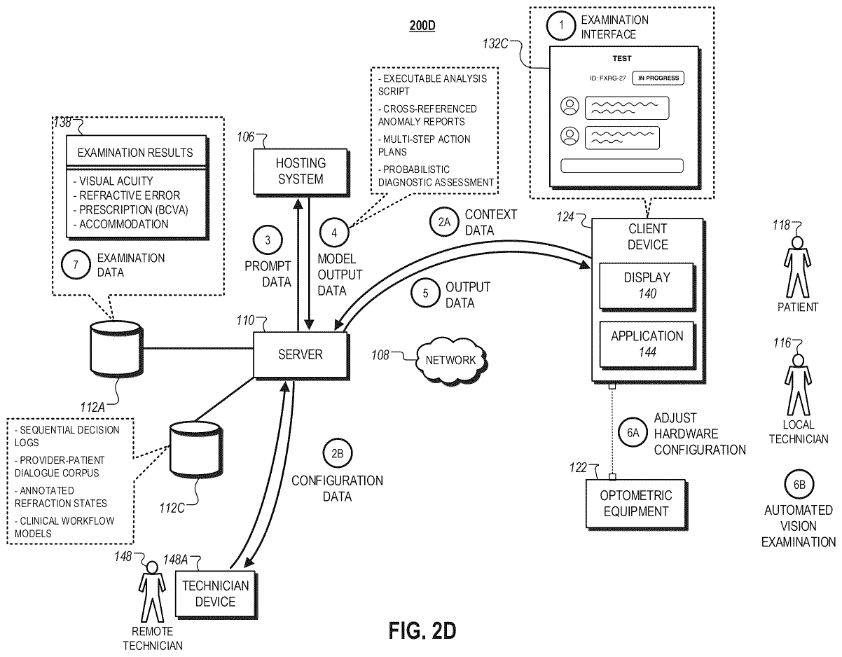

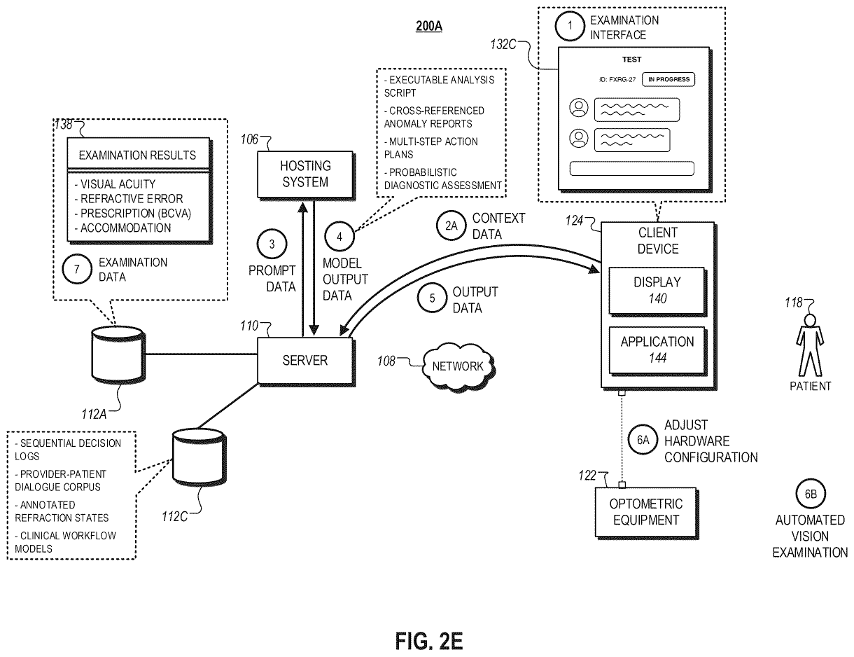

The server 110 functions as the central orchestrator for the system 100 and is configured to provide centralized services for supporting the functionality of the system 100. The server 110 also provides infrastructure support for an application (e.g., application 144 shown in FIGS. 2A-2E) that executes on the client device 124. As illustrated, the server 110 includes several logical modules to manage the examination process: a context analyzer 110A for data ingestion, an orchestrator 110B for managing the core examination logic, and an output processor 110C for generating the machine-readable executable instructions. The server 110 may be configured in various ways, including as a single physical server, a distributed cluster of virtual machines, or as a set of services deployed within a public or private cloud environment, for instance, using a microservices architecture.

To provide the functionality described herein, the server 110 interacts with several key components, including inputs from the data sources 112. The data sources 112 include one or more databases storing different types of data, such as health data 112A, user data 112B, and training data 112C. This data includes historical and contextual information used by the server 110 to personalize and intelligently guide the ML-assisted vision examination. The data sources 112 generally serve as the system's long-term memory, providing the context analyzer 110A with the data needed to build a comprehensive snapshot of the patient and the clinical situation.

The health data 112A includes protected health information (PHI) that is specific to the patient's clinical history and is critical for ensuring the safety and accuracy of the examination. One example of such data is the patient's full prescription history, including the sphere, cylinder, axis, and add power values from all previous examinations. Another example includes detailed logs of patient responses from a previous subjective refraction. In some other examples, the health data 112A includes records of any diagnosed ocular conditions, such as glaucoma, cataracts, or macular degeneration.

The user data 112B includes personally identifiable information (PII) and user-specific preferences that are distinct from the patient's clinical health records. One example of such data is the patient's demographic information, including their date of birth, which the system 100 can use to determine if certain age-related tests are warranted. Another example includes the patient's stated language preference. In some other examples, the user data 112B includes the patient's account settings and communication consent preferences.

The training data 112C includes a specialized corpus of data derived from vision examinations manually performed by expert human providers. This data may be used as reference data to guide prompts provided to the ML models 126 and/or post-process model output data for autonomous examination formats. For example, the training data 112C may specify sequential decision logs recording manual actions taken by a provider. As another example, the training data 112C may include a provider-patient dialogue corpus linking subjective feedback to specific provider actions. In some other examples, the training data 112C includes a collection of annotated refraction states that captures the full state of the optometric equipment 122 when clinical decisions were made.

The server 110 interacts with the hosting system 106 to leverage ML models 126 in vision examination administration. The hosting system 106 provides a managed inference service that receives prompt data from the server 110 and returns machine-generated output used to augment processes for vision examinations. The hosting system 106 may allocate compute resources, schedule model workloads, enforce request quotas, and log usage metrics. Prompt requests may include text segments, video images, or audio data, and response payloads may contain operational guidance for the system 100 to apply.

The hosting system 106 integrates with the system 100 through a set of network-accessible endpoints. The orchestrator 110B authenticates each request with an API key, signs payloads, and posts them to an endpoint path that selects a specific model or model version. The hosting system 106 may reside in a public cloud region, in a dedicated tenancy, or in an on-premise cluster that meets data residency requirements, and configuration flags may allow administrators to choose among these connectivity modes.

One or more ML models 126 reside within the hosting system 106 and/or are hosted by an entity managing the hosting system 106. The ML models 126 implement the inference logic that generates the information used by the system 100. The ML models 126 may be large language models (LLMs), large action models (LAMs), or multimodal (MM) models that accept and emit combinations of text, code, or image embeddings. The hosting system 106 may route traffic to a single model or to an ensemble of models depending on the prompt type and a workspace policy.

The ML models 126 may operate inside the hosting system 106 in containerized runtimes that expose uniform gRPC and REST interfaces. The hosting layer may handle model loading, autoscaling, and the injection of guardrail middleware that checks prompts for policy compliance. Model output is streamed back to the server 110 in an event format that allows for real-time updates to the examination interface.

The server 110 includes various software modules to enable the functionality discussed above. The context analyzer 110A functions as the data ingestion and aggregation module for the server 110, receiving data from the data sources 112 and real-time data from the client device 124 and the provider device 120. The context analyzer 110A synthesizes these various data streams into a single, cohesive, and structured representation of the current examination state, which is then provided as input to the orchestrator 110B.

Upon receiving aggregated context data, the orchestrator 110B manages the logic and decision-making workflows of a vision examination. This involves analyzing the context data to determine a candidate command representing the next logical step in the clinical sequence. Based on this command, the orchestrator 110B generates syntactically correct and contextually rich prompt data for sending to the hosting system 106.

The orchestrator 110B also performs context-preserving anonymization of data before it is included in the prompt data. This anonymization involves specific data transformation processes designed to balance the competing technical requirements of data privacy and the ML models'need for context. The orchestrator 110B is configured to convert specific PII or PHI into clinically relevant but de-identified attributes or tokens, ensuring that the contextual integrity of the data is preserved.

For example, the orchestrator 110B may perform “anonymization by abstraction,” whereby a patient's exact date of birth is transformed into a non-PII, clinically useful age bracket token (e.g., “AGE_BRACKET_40_50”). Similarly, the orchestrator 110B may perform “anonymization by categorization,” where a specific diagnosis from a medical record is transformed into a Boolean clinical history flag (e.g., “HAS_ASTIGMATISM=TRUE”). This specific data transformation process represents a technical improvement to the security and functionality of the system 100.

The orchestrator 110B provides model output data received from the hosting system 106 to the output processor 110C. Operations performed by the output processor 110C are guided by the original candidate command that initiated the query. The post-processing involves parsing the model responses (e.g., by extracting a specific parameter value or an action token) and combining them with the candidate command to generate structured, command-specific executable instructions.

The command-specific executable instructions generated by the output processor 110C are structured data objects that translate analytical results into concrete, machine-readable commands. For example, a common instruction for a spherical power adjustment could be formatted as a data object containing a command identifier such as “ADJUST_SPHERE” and a numerical parameter specifying a value of +0.25, used to increase the phoropter's spherical power.

As another example, an instruction for an astigmatism axis check may contain a command identifier such as “SET_AXIS” and a parameter specifying an angular value of 90. Yet another example is a state-based instruction that contains a command identifier like “SET_LENS_STATE” and a parameter specifying a state such as “occluded” for the left eye. More complex instructions may involve loading an entire set of values simultaneously with a command identifier such as “LOAD_PRESCRIPTION” and a set of parameters for sphere, cylinder, and axis.

The system 100 also includes a variety of user-facing and equipment devices. The provider device 120 is a computing device through which a remote provider 114 administers, supervises, and reviews the ML-assisted vision examination from a remote site 104. The provider device 120 enables the remote provider 114 to transmit high-level clinical commands to the server 110 and to receive real-time data representing the state and results of the examination. The provider device 120 may be, for example, a desktop computer, a laptop computer, a tablet, or a smartphone.

Similarly, a remote technician device (not shown in FIG. 1) is a computing device through which a remote technician administers or assists in the operation of the ML-assisted vision examination from a remote site. The remote technician device (e.g., technician device 148A in FIG. 2A) enables the remote technician to transmit operational or procedural instructions to the server 110 and to receive real-time data. This device may also be a desktop computer, a laptop computer, a tablet, or a smartphone.

The optometric equipment 122 includes the physical, patient-facing diagnostic hardware configured to perform the vision examination. The optometric equipment 122 is controlled by the client device 124, and its optical and/or mechanical components may be adjusted during an examination. The optometric equipment 122 is typically a digital or digitally-controlled phoropter but may also include other devices such as an autorefractor or lensometer with a digital interface.

In some implementations, the optometric equipment 122 represents standard equipment commonly located in diagnostic centers. In such cases, the client device 124 may be coupled to an external actuator system, such as a set of servo motors physically mounted to the conventional phoropter, and software on the client device 124 translates executable instructions from the server 110 into low-level commands that drive the actuators.

In other implementations, the optometric equipment 122 includes special-purpose equipment uniquely designed to function natively with the server 110. For instance, a special-purpose digital phoropter may include integrated digital controllers and a network interface, exposing its own Application Programming Interface (API) to allow an application on the client device 124 to directly transmit command-specific executable instructions to its internal controller.

The client device 124 is a computing device at the examination site 102 that serves as the local interface. The client device 124 enables the capture and transmission of local context data to the server 110 and receives output data representing command-specific executable instructions from the server 110 to control the optometric equipment 122. The client device 124 may be a desktop computer, a tablet computer, a laptop, or a custom-built hardware terminal executing a special-purpose application.

The client device 124 may be used by the patient 118 and/or the local technician 116. In some implementations, where no local technician is present (as in FIGS. 2C and 2E), the client device 124 may be integrated into a self-service kiosk with an integrated graphical user interface and input components to capture patient responses and directly control the integrated optometric equipment. In other implementations, where a local technician 116 is present (as in FIGS. 2A, 2B, 2D), the client device 124 is a dedicated computing terminal used by the technician to facilitate the examination. Through a specialized application, the client device 124 serves as an interface for the technician to manage the workflow, input observations, and relay patient responses to the server 110, as well as to receive and implement instructions from the server 110, either manually or automatically on the optometric equipment 122.

As discussed above, the system 100 may support different types of examination formats. In some implementations, particularly in the augmentation formats (shown in FIGS. 2A, 2B, 2D), the output data provided by the server 110 may not be executed automatically. Instead, the instructions for the optometric equipment 122 may be rendered on the display of the client device 124 for the local technician 116 to implement manually. This hybrid implementation allows the system 100 to provide expert guidance and decision support while a human operator performs the physical hardware interaction. This ensures that some instructions can be implemented by the local technician 116, while other, more routine instructions can be implemented automatically by the optometric equipment 122.

In some other implementations, particularly in the automation formats (shown in FIGS. 2C, 2E), the output data provided by the server 110 may be executed automatically with minimal or no user intervention. In such implementations, application 144 on the client device 124 is configured to receive the output data from the server 110 and, in response, directly cause an adjustment to the hardware configuration of the optometric equipment 122. This establishes a fully automated, closed-loop system in which the patient's response drives the system's internal reasoning and subsequent hardware adjustments without requiring step-by-step confirmation from a human operator. The role of any human participant, such as the remote technician 148 (shown in FIG. 2D), is shifted from direct execution to high-level supervision of the autonomous process.

In some implementations, the server 110 is configured to analyze a patient's speech as part of the process for processing context data and/or model output data. For example, server 110 may determine if a patient's response is one of several expected responses for a given stage of the examination. If the response is expected, server 110 proceeds to generate the command-specific executable instructions. However, if system 100 receives a threshold number of unexpected or ambiguous responses, server 110 may instead generate an output inquiry to repeat an instruction or request clarification from the patient.

Furthermore, system output may not be limited to instructions for the optometric equipment 122. The system may also generate patient-related instructions, which can be provided as an audio response or a visual prompt on the examination interface 132C. These patient-related instructions are distinct from equipment commands and are directed to the patient to facilitate the examination. These instructions may include, for example, an overview of the next test, instructions for the patient to adjust their head position or direct their eye movement, or a specific inquiry about which line or letter they can read on an eye chart.

One example of an examination workflow enabled by system 100 is provided below. The workflow commences with an Initial Visual Acuity (IVA) assessment, orchestrated by the orchestrator 110B. The server 110 provides an initial instruction to the client device 124, causing it to set the optometric equipment 122 to a zero-power state with both eyes of the patient 118 unoccluded. The server 110 also generates a patient-related instruction, such as an audio prompt or a visual cue on the client device 124, asking the patient 118 to read the smallest line of a Snellen eye chart. The patient's 118 verbal response is captured by the client device 124 and transmitted back to the server 110 as context data. The orchestrator 110B processes this response using the ML models 126 via the hosting system 106 to determine if the reading is correct. If the reading is correct, the server 110 may provide further instructions to display a smaller line. Otherwise, the final measurement is stored in the data sources 112 as part of the patient's health data 112A.

Following a binocular assessment, the orchestrator 110B determines the next candidate command is to measure monocular unaided visual acuity. The output processor 110C generates a command-specific executable instruction, such as a state-based instruction with a command identifier like “SET_LENS_STATE” and a parameter specifying “occluded,” to adjust the optometric equipment 122 to occlude the left eye of the patient 118. The process of prompting the patient 118, capturing the verbal response via the client device 124, and processing the response at the server 110 is repeated to determine the visual acuity for the right eye. Subsequently, the orchestrator 110B directs a similar sequence for the left eye by generating instructions to unocclude the left eye and occlude the right eye.

The orchestrator 110B proceeds to a workflow step to measure visual acuity with any existing correction. The context analyzer 110A queries the data sources 112 for relevant lensometry or Auto-Refractor (AR) data within the patient's health data 112A. If lensometry data exists, the server 110 generates executable instructions, such as a “LOAD_PRESCRIPTION” command, to load the patient's 118 existing prescription into the optometric equipment 122. The visual acuity may be measured again (binocularly, monocularly) with results stored in health data 112A. If AR data is available, this process is repeated using the AR data to configure the optometric equipment 122. If no such historical data is found in data sources 112, the orchestrator 110B may be configured to skip these steps.

In the exemplary workflow discussed above, system 100 is used to perform a subjective refraction. In this example, the orchestrator 110B initiates by loading the patient's AR data from health data 112A into the optometric equipment 122. Based on a predefined clinical rule, the orchestrator 110B determines a candidate command to “fog” the tested eye to relax accommodation. The output processor 110C generates an instruction, such as “ADJUST_SPHERE” with a parameter of +1.00, which is sent to the client device 124 to adjust the optometric equipment 122. The server 110 also initiates an interactive loop, including prompting the patient 118 to read an eye chart and evaluating the response. If acuity does not improve, an additional +0.25 sphere power may be added to establish a reliable starting point for the refraction.

The orchestrator 110B determines the candidate command is to perform a Jackson Cross Cylinder (JCC) test to find the cylinder axis. If the context data from health data 112A indicates no cylinder, the server 110 generates instructions to introduce a probing cylinder (e.g., −0.50) and a compensating sphere adjustment. The server 110 also instructs the client device 124 to display an astigmatic dots chart and present the first JCC lens position. A verbal prompt, such as “Is it better one?”, is generated. The patient's 118 response (e.g., “one is better”) is captured as context data and transmitted to the server 110.

The orchestrator 110B generates prompt data for the hosting system 106 by querying the ML models 126 for an appropriate adjustment. The hosting system 106 returns model output data containing a parameter value (e.g., +10 degrees). The output processor 110C parses this value and generates a “SET_AXIS” instruction. This interactive loop continues, with the server 110 halving the adjustment value each time the patient's 118 preference reverses, until the model output data, based on a patient response of “about the same,” indicates the axis is found.

Once the axis is finalized, the orchestrator 110B determines the next candidate command is to check the JCC for cylinder power. This follows a similar interactive loop where the server 110 provides instructions to present two different JCC power options, each with a verbal prompt. The patient's 118 response is used to generate prompt data for the hosting system 106, and the returned model output data contains a parameter value for the adjustment (e.g., −0.25 cylinder). During this process, the server 110 may also apply an internal clinical rule to maintain the spherical equivalent, causing the output processor 110C to generate an additional instruction to adjust the sphere power (e.g., by +0.25D) for every −0.50D of cylinder power added. This loop may repeat until the patient's 118 response indicates the optimal power has been found.

With the cylinder correction finalized, the orchestrator 110B may perform a final sphere refinement using a red/green duochrome test. This step may be skipped if the patient's 118 acuity is already at a predetermined threshold (e.g., 20/20). Otherwise, the server 110 instructs the client device 124 to display the appropriate chart and prompts the patient 118 (e.g., “Are the letters sharper on the red side or the green side?”). The patient's 118 response (e.g., “red”) is included in prompt data to the hosting system 106. The ML models 126 return model output data containing the corresponding sphere power adjustment (e.g., −0.25), which the output processor 110C uses to generate the executable instruction. This process continues until the patient's response indicates equilibrium has been reached.

Upon completion of the subjective refraction for the first eye, the workflow discussed above (including axis, cylinder, and sphere refinement) is repeated for the other eye of the patient, as managed by the orchestrator 110B. After both eyes are refracted, the server 110 conducts a Final Distance Visual Acuity (FDVA) test by loading the newly determined prescription into the optometric equipment 122 and measuring the final acuity. As a final validation, the server 110 may generate instructions to load the patient's 118 old prescription (from health data 112A) and then the new prescription, prompting the patient 118 for their subjective preference, with the choice being recorded by the server 110.

For applicable patients, the orchestrator 110B may initiate a conditional Add Power Test for near vision. This additional workflow is triggered if context data from user data 112B (e.g., patient's age is over 40) and/or health data 112A (e.g., a history of presbyopia) indicates a need. The server 110 instructs a local technician 116 via the client device 124 to lower a reading rod. An initial add power is loaded into the optometric equipment 122, and the server 110 begins an iterative process of increasing the power in +0.25D increments, prompting the patient 118 to read a near-vision chart at each step. The test concludes when the model output data, derived from the patient's 118 responses, indicates that near visual acuity is no longer improving.

The vision examination concludes when the orchestrator 110B determines all workflow steps are complete. The server 110 provides final instructions to the client device 124, such as instructing the local technician 116 to raise the reading rod and displaying a completion message to the patient 118. Data gathered during the automated refraction is compiled by the server 110 and stored as the final examination results in health data 112A, ready for asynchronous review and validation by a remote provider 114 using a provider device 120.

FIG. 2A is a conceptual diagram of an exemplary system 200A for administering ML-assisted vision examinations with a format that includes a local technician, a remote technician, and a remote provider. The system 200A may include one or more servers or computers, such as server 110, connected locally or over a network to various devices.

The system 200A includes a network 108, which may be, for example, a local network, a Wi-Fi network, an intranet, or an internet connection that enables communication between the server 110, a hosting system 106, a client device 124, a provider device 120, and a technician device 148A. In some implementations, the system 200A may be performed by a cloud computing system over the network 108.

FIG. 2A illustrates various operations in stages (1) through (7), which may be performed in the sequence indicated or in another sequence. This format represents a highly collaborative, human-assisted examination format where a patient 118 and a local technician 116 are physically present at the examination site, while a remote provider 114 and a remote technician 148 participate from remote locations to conduct the examination. In this examination format, system 200A leverages ML models 126 as an intelligent assistant, processing patient responses and providing decision support to remote human operators.

During stage (1), the client device 124, which may be a personal computer, tablet, or specialized medical interface, presents an examination interface 132C on its display 140. The local technician 116 may use this interface to initiate the test and assist the patient 118. The patient 118 interacts with the system 200A, for instance by providing verbal responses to prompts shown on the interface. An application 144 running on the client device 124 manages the user interface and communication with the server 110.

During stages (2A), (2B), and (6B), the server 110 receives context data from multiple external data sources to establish the current state of the vision examination. At stage (2A), the client device 124 transmits context data to the server 110, which may include digitized audio of the patient's verbal responses. At stages (2B), and (6B), the remote technician 148 and remote provider 114 use their respective devices, technician device 148A and provider device 120, to send configuration data and instructions to administer the vision examination, which forms part of the overall context data.

During stage (3), the server 110 generates and provides prompt data to the hosting system 106. To enable effective ML-based augmentation, this prompt data includes not only the patient's raw response data from stage (2A) but also crucial context, such as the specific test being performed (e.g., “JCC axis check”) and options that may have been presented to the patient. This structured prompt allows the ML models 126 associated with the hosting system 106 to perform a contextually relevant analysis rather than a generic interpretation.

During stage (4), the hosting system 106 processes the prompt data and returns model output data to the server 110. In the examination format depicted in FIG. 2A, the primary function of ML models 126 is to augment the human operators'perception and interpretation of subjective patient data. The model output data is therefore advisory and analytical, designed to supplement rather than replace human judgment. For example, a Response Validation Token may classify the patient's subjective response as “clear,” “ambiguous,” or “unexpected,” which saves the operator from having to make that judgment call. As another example, a clinical recommendation may suggest a specific phoropter adjustment (e.g., “+0.25 sphere”) with an associated confidence score, guiding less experienced technicians. An anomaly flag may be generated if the ML models 126 detect, for instance, a patient response that is statistically inconsistent with previous responses, and thereby alert the operator to a potential issue. A state summary object may distill a series of complex responses into a simple, human-readable summary for the provider's review.

During stage (5), the server 110 parses the assistive model output data. The server 110 may present this analysis on the interfaces of the provider device 120 and/or technician device 148A. An example of the analysis includes displaying a recommended action with buttons for the human operator to “accept” or “reject” the recommended action. The operator's final decision is transmitted back to the server 110 (as part of stage 6B). The server 110 generates the final output data, which represents the command-specific executable instructions corresponding to the operator's confirmed decision. This output data is then provided to the client device 124.

During stage (6A), the application 144 on the client device 124 receives the output data and causes an adjustment to the hardware configuration of the optometric equipment 122. During stage (7), the server 110 stores the resulting examination data in a database 112A, creating a record of the examination results 138 for final review by the remote provider 114.

FIG. 2B is a conceptual diagram of an exemplary system 200B for administering ML-assisted vision examinations with a format that includes a local technician and a remote provider. The system 200B may include one or more servers or computers, such as server 110, connected locally or over a network 108 to various devices, including a hosting system 106, a client device 124, and a provider device 120.

FIG. 2B illustrates various operations in stages (1) through (7), which may be performed in the sequence indicated or in another sequence. This represents an examination format in which a patient 118 and a local technician 116 are physically present, and a remote provider 114 acts as the sole clinical authority. A key challenge in this examination format is that the remote provider 114, while clinically proficient, may not be an expert in operating the specific user interface or API of the application 144. The system 200B addresses this by using ML models to augment the provider's workflow, translating their clinical goals into specific software operations.

During stage (1), the client device 124 presents an examination interface 132C on its display 140. During stages (2) and (6B), the server 110 receives context data. At stage (2), this includes context data, such as the patient's 118 responses. At stage (6B), the remote provider 114 uses the provider device 120 to transmit a high-level command to administer the vision examination. This command represents a clinical goal (e.g., “check for astigmatism”) rather than a specific software command.

During stage (3), the server 110 generates and provides prompt data to the hosting system 106. This prompt data includes the provider's clinical goal and the current examination state. This context is important for ML models 126 to determine the most efficient sequence of software actions required to fulfill the provider's intent.

During stage (4), the hosting system 106 returns model output data specifically designed to facilitate the operation of the software. Here, the ML model 126's function evolves from clinical analysis (depicted in FIG. 2A) to operational expertise. The ML models 126 act as an expert user of the application 144, augmenting the provider by translating their high-level clinical intent into low-level software commands. For example, in response to a “check astigmatism” goal, the ML models 126 may generate a pre-formatted API call, which is a syntactically correct command string that the server 110 can use to directly control the application 144. Alternatively, the ML models 126 may output a workflow macro, which is a script that automates a series of otherwise manual steps. The ML models 126 can also provide UI-based navigation suggestions to guide the provider, or parameter autofill data to pre-populate fields with optimal starting values.

During stage (5), the server 110 parses the operational model output data. Depending on the output type, the server 110 might present the data to the provider for one-click execution (e.g., a “Run Macro” button) or directly use the data to generate the final output data. This output data represents the command-specific executable instructions for the client device 124.

During stage (6A), the application 144 on the client device 124 receives the output data and causes an adjustment to the hardware configuration of the optometric equipment 122. During stage (7), the server 110 stores examination data in a database 112A, making the examination results 138 available for the remote provider 114 to review and finalize.

FIG. 2C is a conceptual diagram of an exemplary system 200C for administering ML-assisted vision examinations with a format that includes only a remote provider. The system 200C may include one or more servers or computers, such as server 110, connected locally or over a network 108 to various devices, including a hosting system 106, a client device 124, and a provider device 120.

FIG. 2C illustrates various operations in stages (1) through (7), which may be performed in the sequence indicated or in another sequence. This format represents a high-augmentation model where a patient 118 interacts directly with the system, and a remote provider 114 supervises without providing step-by-step commands. This configuration relies on an “agentic” use of the ML models, where the models autonomously determine the examination's progression based on a logical clinical workflow and the patient's real-time responses. The remote provider 114 monitors the automated process and can intervene if an unexpected situation arises.

During stage (1), the client device 124 presents an examination interface 132C on its display 140. At stage (2A), the client device 124 transmits context data, which primarily consists of the patient's 118 responses. At stage (6B), the provider device 120 may transmit high-level commands to administer the vision examination, such as “start” or “pause,” which control the overall state of the autonomous agent.

During stage (3), the server 110, having analyzed the patient's last response, autonomously determines the next action and generates corresponding prompt data for the hosting system 106. For example, if the server's internal state machine indicates it is performing a sphere refinement and the patient's response was “the letters on the red side are sharper,” the prompt will query the ML model for the specific parameter value needed for the next adjustment.

During stage (4), the hosting system 106 returns model output data that is agentic and directly machine-executable by the server 110. In this format, the ML models'role shifts from an enabler for a human operator to the primary agent driving the examination workflow. The models are used here to execute a pre-trained clinical logic, outputting definitive commands instead of advice. For example, a direct action token such as ADJUST_SPHERE instructs the server on what to do, while a separate parameter value output like “−0.25” provides the specific value for that action. A state-transition command can be used to instruct the server's workflow engine to conclude one test, and for complex state changes, the model may output a complete hardware-configuration vector.

During stage (5), the server 110 parses the agentic model output data. For example, it may receive a direct action token and a corresponding parameter value. The server then combines these to generate the final output data containing the fully formed, command-specific executable instructions. This process is fully automated for routine steps and does not require confirmation from the remote provider 114. During stage (6A), the application 144 on the client device 124 receives the output data and causes the autonomous adjustment to the hardware configuration of the optometric equipment 122. During stage (7), the server 110 stores the examination data, compiling the examination results 138 for the provider's final review and approval.

FIG. 2D is a conceptual diagram of an exemplary system 200D for administering ML-assisted vision examinations with a format that includes a local technician and a remote technician. The system 200D may include one or more servers or computers, such as server 110, connected locally or over a network 108 to various devices, including a hosting system 106, a client device 124, and a technician device 148A.

FIG. 2D illustrates various operations in stages (1) through (7), which may be performed in the sequence indicated or in another sequence. This format represents an advanced augmentation model where there is no real-time provider involvement. A patient 118, a local technician 116, and a remote technician 148 are present, with the latter initiating an automated vision examination (6B). The core of this model is the use of machine learning to perform complex reasoning and assessment tasks that would traditionally be handled by a provider, thus augmenting the capabilities of the technician.

To enable this high-level reasoning, the ML models of the hosting system 106 are trained on training data 112C that is accessible by the server 110. This database contains data derived from a large corpus of examinations performed by expert human clinicians. As illustrated, this data may include sequential decision logs that map patient responses to provider actions, a provider-patient dialogue corpus to understand subjective language, annotated refraction states that label specific clinical scenarios, and clinical workflow models that describe standard and exception-based examination paths. This training endows the models with the ability to emulate a provider's diagnostic and procedural logic.

During stages (1) and (2A-B), the examination is initiated by the remote technician 148, and context data (including the patient's 118 responses) is sent to the server 110. At stage (3), the server 110 generates prompt data for the hosting system 106. This prompt might request a comprehensive analysis, including the patient's real-time responses and their historical data from database 112A.

During stage (4), the hosting system 106 returns model output data that is the product of this advanced reasoning. The ML model 126 function evolves beyond executing a workflow to emulating a provider's diagnostic reasoning by synthesizing disparate data. The ML models 126 are specifically used to analyze the full clinical picture by correlating real-time responses with historical data.

For example, the model may generate an executable analysis script that instructs the server 110 to perform a specific correlation, such as comparing the rate of myopia progression from historical data against the current findings. It can produce a cross-referenced anomaly report that flags an unusual patient response and points to a specific entry in their past medical record that might explain it. In complex situations, the model might output a multi-step action plan to resolve an ambiguous clinical finding, or a probabilistic diagnostic assessment providing a list of potential underlying conditions. During stage (5), the server 110 parses this sophisticated model output data and translates it into a sequence of executable instructions in its output data. During stage (6A), the client device 124 executes these instructions, adjusting the optometric equipment 122. During stage (7), the server 110 stores the examination data, including the AI-generated reports and assessments, as examination results 138 for a provider's final, asynchronous sign-off.

FIG. 2E is a conceptual diagram of an exemplary system 200E for administering a fully autonomous vision examination. The system 200E may include one or more servers or computers, such as server 110, connected locally or over a network 108 to various devices, including a hosting system 106 and a client device 124.

FIG. 2E illustrates various operations in stages (1) through (7), which may be performed in the sequence indicated or in another sequence. This format represents a fully automated model in which ML models 126 replace the real-time functions of a human provider and technician. A patient 118 interacts directly with the system, and a local technician 116 may be present only to initiate the automated vision examination (6B) and provide initial setup support.

The capability for full automation is specifically enabled by the ML models 126 of the hosting system 106 having been trained on training data 112C. This database contains a comprehensive corpus of examinations performed by expert human clinicians. By training on sequential decision logs and clinical workflow models, the ML models 126 learn to navigate the entire refraction process, including standard procedures and exception handling. Training on a provider-patient dialogue corpus allows the model to accurately interpret a wide range of subjective patient language, while training on annotated refraction states enables it to recognize specific clinical scenarios and their resolutions.

During stages (1) and (2A), the patient 118 interacts with the examination interface 132C, and the client device 124 transmits context data containing the patient's direct responses to the server 110.

During stage (3), the server 110 autonomously generates prompt data for the hosting system 106, querying the ML models 126 to perform the next required clinical reasoning task.

During stage (4), the hosting system 106 returns model output data that represents the definitive outcome of a complex reasoning process. In this final evolution, the ML model 126 function is to act as a fully autonomous clinical decision-maker, entirely replacing the real-time judgment of a human provider. The model is used to generate an executable analysis script to perform analyses a provider would typically do mentally. The model autonomously generates a cross-referenced anomaly report and uses this report to modify its own subsequent actions without human confirmation. The model may output a multi-step action plan, which is a self-determined sequence of hardware adjustments. At the conclusion of the data gathering, the model generates a probabilistic diagnostic assessment, which is a clinical conclusion that becomes a primary component of the final report.

During stage (5), the server 110 parses this definitive model output data and translates it into the corresponding sequence of command-specific executable instructions, which it provides as output data to the client device 124. This closed loop of patient interaction, ML-assisted reasoning, and hardware adjustment repeats without any human intervention. During stage (6A), the application 144 causes the autonomous adjustments to the optometric equipment 122. During stage (7), upon autonomous completion, the server 110 stores the examination data, including the AI-generated reports and assessments, as final examination results 138, which are then queued for a provider's asynchronous review and final validation.

In some implementations, the one or more ML models 126 of the hosting system 106 can be deployed in various architectural configurations to provide the functionalities described in FIGS. 2A-2E. For example, the hosting system 106 may be a third-party service provider, and the server 110 communicates with it over the network 108 via a secure API. In some implementations, the one or more ML models 126 may be deployed directly on the server 110, creating a more integrated system. In yet other implementations, the one or more ML models 126 may be distributed, such that certain portions of a model reside on the hosting system 106 while other portions reside on the server 110, allowing for optimized processing of different tasks.

The level of automation or augmentation provided by the system, as illustrated by the distinct formats in FIGS. 2A-2E, is not necessarily static for a given examination. In some implementations, the server 110 is configured to dynamically adjust its reliance on the one or more ML models 126 based on one or more criteria associated with the patient 118. For example, these criteria may include demographic parameters such as age, vision health, a history of vision impairment, or prior refractive errors. Each demographic criterion may have an associated demographic threshold that the server 110 uses to determine the appropriate level of ML involvement.

For example, the server 110 may be configured to reduce its reliance on the one or more ML models 126 for patients who meet certain demographic thresholds, thereby ensuring a greater degree of human oversight for more complex or sensitive cases. Where age is a demographic criterion and the patient 118 is above an age threshold (e.g., 90 years old), the server 110 may reduce its reliance on the ML models to a first degree, for instance by shifting from a fully agentic model (as in FIG. 2C) to a more assistive, human-in-the-loop model (as in FIG. 2A). If a second demographic criterion, such as a history of significant vision impairment, also exceeds a threshold, the server 110 may reduce its reliance to a second, greater degree, requiring more explicit human confirmation for each step. Alternatively, if a demographic threshold is not satisfied for a given demographic criterion, the server 110 may be configured to increase its reliance on the one or more ML models 126 to perform the vision examination. For example, if the age of the patient 118 (e.g., 30 years old) fails to satisfy an age threshold, the server 110 can increase its reliance on the ML models. Where the patient's age fails to satisfy the age threshold and their vision status (e.g., excellent vision health) also fails to satisfy a vision status threshold, the reliance on the one or more ML models 126 can increase to a second, higher base degree. Accordingly, in some implementations, the system is more likely to utilize a fully autonomous examination format, such as that shown in FIG. 2E, for younger patients with no history of significant vision impairments.

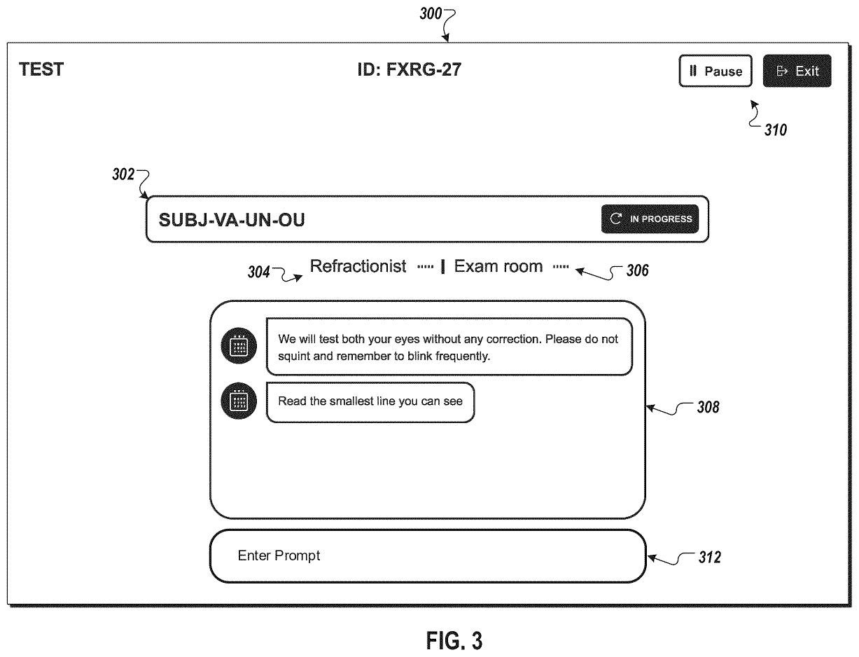

FIG. 3 is a conceptual diagram of an exemplary interface for facilitating ML-assisted vision examinations. The interface 300 may be implemented on the display 140 of the client device 124. The interface 300 includes one or more action buttons 310 that may enable a local technician, remote technician, or remote provider to start, pause, or end the vision examination or a particular segment of the vision examination.

The interface 300 includes an indication 302 of the type of test being administered during the vision examination. As will be explained in more detail below, the vision examination can include a variety of tests, such as embedded refraction, visual-acuity, sphere, and JCC tests. The indication 302 may also display a status, such as “IN PROGRESS,” which may indicate that a particular test is active, that the system is awaiting user input, or that it is processing information.

An administrator tab 304 may indicate which entity, such as a local technician or a remote provider, is currently conducting the vision examination or a particular segment thereof. A station indicator 306 may specify a point in the vision examination process. For example, the station indicator 306 may display “Exam room” to communicate to the patient that the examination is in progress.

The interface 300 includes a prompt area 308 that displays a plurality of prompts related to the vision examination. For example, a given test may include one or more instructions that request input from the patient, such as “We will test both your eyes without any correction. Please do not squint and remember to blink frequently,” and “Read the smallest line you can see.” The interface 300 may also include an input field 312, which can be used by a technician or provider to enter prompts or other data into the system.

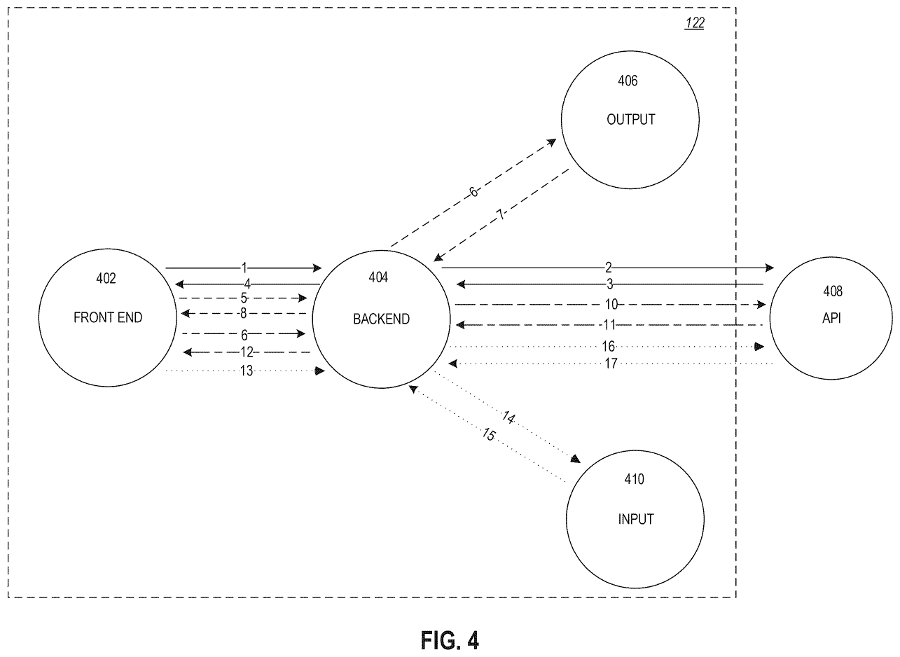

FIG. 4 is a block diagram of exemplary communication flows involved in ML-assisted vision examinations. A front end 402 (which may be implemented by the application 144 on the client device 124) communicates with a backend 404 (which may be implemented by the server 110). The backend 404 orchestrates communications between the front end 402, an output component 406, an input component 410, and an API 408.

The examination process may be initiated at operation 1, where the front end 402 transmits a start instruction to the backend 404. The backend 404 may forward this instruction to the API 408 at operation 2. At operation 3, the API 408 may generate and transmit a corresponding instruction back to the backend 404, which then forwards the instruction to the front end 402 at operation 4.

At operation 5, the front end 402 may determine an instruction type and generate a corresponding indicator that is communicated to the backend 404. For example, the instruction type may indicate that an audio prompt should be presented to the patient. At operation 6, the backend 404 processes this instruction type and transmits a corresponding audio instruction to an output component 406, which may represent an audio speaker of the client device 124.

At operation 7, the output component 406 may generate an audio output, and a confirmation of this action can be transmitted back to the backend 404. This confirmation may be forwarded to the front end 402 at operation 8. Upon receiving the confirmation, the front end 402 may transmit a new instruction type to the backend 404 at operation 9. At operation 10, the backend 404 may generate an execution instruction and transmit it to the API 408. The API 408 may then generate and transmit an acknowledgement back to the backend 404 at operation 11.

At operation 12, the acknowledgement from the API 408 is received at the backend 404 and forwarded to the front end 402. At operation 13, the front end 402 may generate a new instruction type indicating that the system should now acquire a response from the user. At operation 14, the backend 404 transmits an instruction to an input component 410, which may represent a microphone of the client device 124. At operation 15, the input component 410 acquires the user's verbal response, digitizes it, and transmits it to the backend 404.

In response to receiving the digitized verbal response, the backend 404 generates an instruction to proceed to the next step of the examination, which is transmitted to the API 408 at operation 16. The API 408 may determine if a next instruction exists in the examination sequence. If a next instruction exists, the API 408 generates this instruction and transmits it to the backend 404 at operation 17. This process may then loop back to operation 3 to continue the examination sequence.

In some implementations, a pause instruction may be received at the front end 402 from a user. This pause instruction may be transmitted to the backend 404 to temporarily suspend the generation of new instructions or the processing of user inputs. This allows a user, such as a technician or provider, to halt the automated or augmented examination flow as needed.

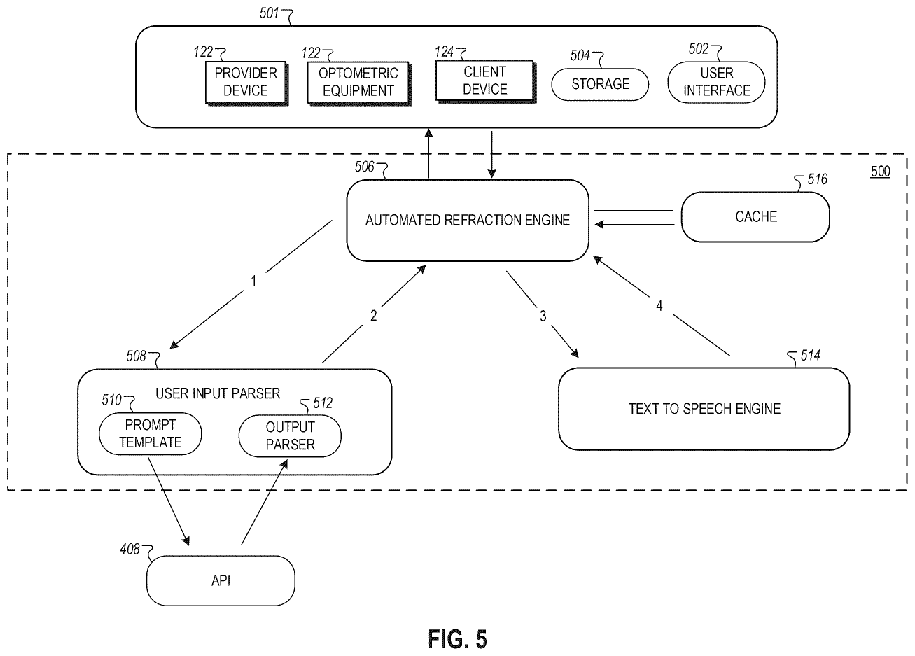

FIG. 5 is a block diagram of an exemplary logical architecture 500 for processing data associated with ML-assisted vision examinations. The architecture 500 includes an automated refraction engine 506, a user input parser 508, a text-to-speech engine 514, and a cache 516. In some implementations, the logical architecture 500 may be performed by the server 110, with its components distributed across various modules of the server.

The logical architecture 500 is configured to communicate with a plurality of devices 501. These devices may include, for example, a provider device 120, the optometric equipment 122, and the client device 124. Each of these devices may provide a user interface 502 and may have access to local or remote storage 504.

The automated refraction engine 506 acts as a central orchestrator for the data flows within the architecture 500. For example, upon receiving data indicative of patient audio from one of the devices 501, the automated refraction engine 506 transmits this data to the user input parser 508 at data stream 1. In some implementations, the data indicative of patient audio may have been previously converted to text.

The user input parser 508 is responsible for interacting with the ML models 126. As shown, the user input parser 508 includes a prompt template 510 and an output parser 512, both of which communicate with an API 408. The prompt template 510 may structure the received patient data into a syntactically correct prompt, which is then sent to the one or more ML models via the API 408. The API 408, which may be an interface to the hosting system 106, returns model output data to the output parser 512. At data stream 2, the output parser 512 provides a structured and parsed input, derived from the model output data, back to the automated refraction engine 506.

The automated refraction engine 506 may process the parsed input and determine that an audio response is required for the patient. At data stream 3, the engine transmits data indicative of a command and a state to the text-to-speech engine 514. At data stream 4, the text-to-speech engine 514 generates data indicative of an audio response and transmits it back to the automated refraction engine 506. The automated refraction engine 506 then provides this audio response data to the appropriate device from the plurality of devices 501. The cache 516 may be used to store frequently accessed data, such as common audio responses or prompt structures, to improve system performance.

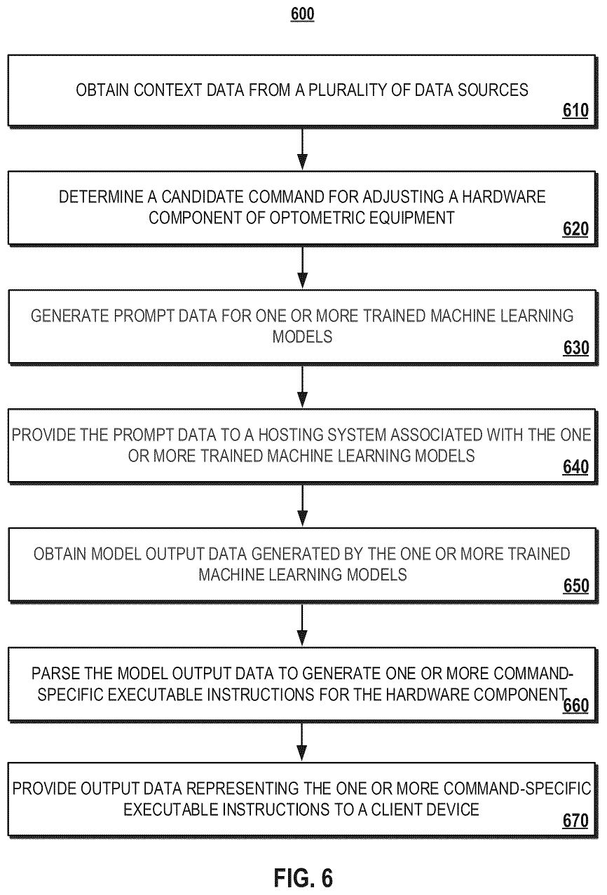

FIG. 6 is a flow chart illustrating an example process 600 of enabling ML-assisted vision examinations. In general, method 600 includes the operations of obtaining context data from a plurality of data sources (610), determining a candidate command for adjusting a hardware component of optometric equipment (620), generating prompt data for one or more trained ML models (630), providing the prompt data to a hosting system associated with the one or more trained ML models (640), obtaining model output data generated by the one or more trained ML models (650), parsing the model output data to generate one or more command-specific executable instructions for the hardware component (660), and providing output data representing the one or more command-specific executable instructions to a client device (670).

In more detail, process 600 includes receiving context data from a plurality of external data sources (610). For example, the context analyzer 110A of the server 110 receives historical vision examination data for a patient from health data 112A and user data 112B, along with data representing a current state of a vision examination being administered at the examination site 102. This data may be received over the network 108 from various devices, such as the client device 124.

In some implementations, the context data further comprises input data from the patient, which is received by the server 110 from a remote patient device during the administration of the vision examination.

Process 600 includes determining a candidate command for adjusting a hardware component of optometric equipment (620). For example, the orchestrator 110B of the server 110 analyzes the current state of the vision examination included in the context data to determine the next logical action in a clinical workflow. In some implementations, the candidate command is determined based on the historical vision examination data and the input data from the patient.

In some implementations, the optometric equipment 122 is a phoropter. In such implementations, the candidate command is a command to perform an adjustment for the phoropter, such as adjusting a spherical lens power, adjusting a cylindrical lens power, adjusting an axis of a cylindrical lens, adjusting a position of a JCC lens, adjusting an add power, and adjusting an occluded state of a lens.

Process 600 includes generating prompt data for one or more trained ML models (630). For example, the orchestrator 110B generates the prompt data based on the candidate command determined at operation 620. The prompt data specifies a natural language query of the candidate command for a proposed change to a configuration of the hardware component.

In some implementations, generating the prompt data comprises structuring the query to integrate the candidate command, the historical vision examination data, the current state of the vision examination, and the input data from the patient.

Process 600 includes providing the prompt data to a hosting system (640) and obtaining model output data (650). For example, the server 110 provides the prompt data to the hosting system 106 over the network 108. The hosting system 106 processes the prompt using one or more ML models 126 and returns the resulting model output data to the server 110.

Process 600 includes parsing the model output data to generate one or more command-specific executable instructions (660). For example, the output processor 110C of the server 110 parses the received model output data based on the original candidate command to generate the executable instructions.

In some implementations, the natural language query is configured to elicit a value corresponding to the candidate command, parsing the model output data comprises extracting the value, and the one or more command-specific executable instructions are generated based on the candidate command and the value.

In some implementations, parsing the model output data comprises determining a degree of similarity between the model output data and one or more stored responses; in response to determining that the degree of similarity satisfies a similarity threshold, generating the one or more command-specific executable instructions; and in response to determining that the degree of similarity does not satisfy the similarity threshold, generating an output inquiry.

In some implementations, in response to generating the output inquiry, the method further comprises incrementing a counter and, in response to determining that the counter satisfies a count threshold, establishing a network connection between the client device 124 and a remote provider device.

Process 600 includes providing output data representing the one or more command-specific executable instructions for output (670). For example, the output processor 110C of the server 110 provides the final output data to the client device 124. This output data is formatted such that, when received, it causes the client device 124 to initiate a change to a configuration of the hardware component of the optometric equipment 122.

In some implementations, the change to the configuration comprises updating an eye chart displayed on a graphical user interface of the client device 124. In some implementations, the client device 124 comprises a phoropter control device, each of the one or more command-specific executable instructions specifies (i) a command identifier for the adjustment and (ii) a parameter that quantifies a degree of the adjustment, and the output data is formatted for an application programming interface (API) of the phoropter control device.

In some implementations, in addition to generating instructions for the optometric equipment, the server 110 may also generate an audio response for the patient. The audio response may comprise a verbal prompt corresponding to the candidate command, and the server 110 provides this audio response for output to the remote patient device. In further implementations, the server 110 may first receive data indicative of speech from the patient and convert it to text, wherein the input data from the patient comprises this text. In yet further implementations, generating the audio response may comprise determining one or more waveforms, or a pitch and a rhythm, from the data indicative of speech and using this information to generate one or more response waveforms. This process may be further refined by obtaining data indicative of phonemes from the hosting system 106 and applying the phonemes to modulate the one or more response waveforms.

Implementations of the subject matter and the functional operations described in this specification may be implemented in digital electronic circuitry, in tangibly-embodied computer software or firmware, in computer hardware, including the structures disclosed in this specification and their structural equivalents, or in combinations of one or more of them. Implementations may be implemented as one or more computer programs, i.e., one or more modules of computer program instructions encoded on a tangible non-transitory program carrier for execution by, or to control the operation of, data processing apparatus. Alternatively, or in addition, the program instructions can be encoded on an artificially generated propagated signal, e.g., a machine-generated electrical, optical, or electromagnetic signal, which is generated to encode information for transmission to suitable receiver apparatus for execution by a data processing apparatus. The computer storage medium can be a machine-readable storage device, a machine-readable storage substrate, a random or serial access memory device, or a combination of one or more of them. The computer storage medium is not, however, a propagated signal.

The term “data processing apparatus” encompasses all kinds of apparatus, devices, and machines for processing data, including by way of example a programmable processor, a computer, or multiple processors or computers. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, subprograms, or portions of code. A computer program can be deployed to be executed on one computer or on multiple computers that are located at one site or distributed across multiple sites and interconnected by a communication network.

Computers suitable for the execution of a computer program include, by way of example, can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magnetooptical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magnetooptical disks; and CDROM and DVD-ROM disks. The processor and the memory can be supplemented by, or incorporated in, special purpose logic circuitry.

Implementations of the subject matter described in this specification can be implemented in a computing system that includes a backend component, e.g., as a data server, or that includes a middleware component, e.g., an application server, or that includes a frontend component, e.g., a client computer having a graphical user interface or a Web browser through which a user can interact with an implementation of the subject matter described in this specification, or any combination of one or more such backend, middleware, or frontend components. The components of the system can be interconnected by any form or medium of digital data communication, e.g., a communication network. Examples of communication networks include a local area network (“LAN”) and a wide area network (“WAN”), e.g., the Internet.

While this specification contains specific implementation details, these should not be construed as limitations on the scope of any invention or of what may be claimed, but rather as descriptions of features that may be specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the implementations described above should not be understood as requiring such separation in all implementations, and it should be understood that the described program components and systems can generally be integrated together in a single software product or packaged into multiple software products.

Devices and techniques for implementing augmented vision examination using one or more ML models is disclosed. Particular implementations of the subject matter have been described. Other implementations are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

A number of implementations have been described. Nevertheless, it will be understood that various modifications may be made without departing from the spirit and scope of the invention. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

What is claimed is:

1. A method implemented by one or more computing devices, the method comprising:
obtaining, from a plurality of external data sources, context data, wherein the context data comprises (i) historical vision examination data for a patient received from one or more databases, (ii) a current state of a vision examination being administered for the patient;
determining a candidate command for adjusting a hardware component of optometric equipment based on the current state of the vision examination;
generating prompt data for one or more trained machine learning models based on the candidate command, wherein the prompt data specifies a natural language query of the candidate command for a proposed change to a configuration of the hardware component;
providing the prompt data to a hosting system associated with the one or more trained machine learning models;
obtaining, from the hosting system, model output data generated by the one or more trained machine learning models in response to providing the prompt data;
parsing the model output data based on the candidate command to generate one or more command-specific executable instructions for the hardware component; and
providing output data representing the one or more command-specific executable instructions for output, wherein the output data, when received by a client device, causes the client device to initiate a change to a configuration of the hardware component.

2. The method of claim 1, wherein:
the context data further comprises input data from the patient received from a remote patient device during the administration of the vision examination; and
the candidate command is determined based on the historical vision examination data and the input data from the patient.

3. The method of claim 2, wherein generating the prompt data comprises structuring the query to integrate the candidate command, the historical vision examination data, the current state of the vision examination, and the input data from the patient.

4. The method of claim 3, wherein:
the natural language query is configured to elicit a value corresponding to the candidate command;
parsing the model output data comprises extracting the value; and
the one or more command-specific executable instructions are generated based on the candidate command and the value.

5. The method of claim 2, comprising:
receiving, from the remote patient device, data indicative of speech from the patient; and
converting the data indicative of speech to data indicative of text, wherein the input data from the patient comprises the data indicative of text.

6. The method of claim 2, further comprising:
generating an audio response for the patient, wherein the audio response comprises a verbal prompt corresponding to the candidate command; and
providing the audio response for output to the remote patient device.

7. The method of claim 6, further comprising:
receiving, from the remote patient device, data indicative of speech from the patient; and
wherein generating the audio response comprises:
determining one or more waveforms from the data indicative of speech; and
generating one or more response waveforms based on the determined waveforms, wherein the audio response comprises the one or more response waveforms.

8. The method of claim 6, further comprising:
receiving, from the remote patient device, data indicative of speech from the patient; and
wherein generating the audio response comprises:
determining a pitch and a rhythm of the data indicative of speech; and
generating one or more response waveforms based on the determined pitch and rhythm, wherein the audio response comprises the one or more response waveforms.

9. The method of claim 8, wherein generating the one or more response waveforms further comprises:
obtaining, from the hosting system, data indicative of phonemes corresponding to the determined pitch and rhythm; and
applying the data indicative of phonemes to modulate the one or more response waveforms.

10. The method of claim 1, wherein:
the optometric equipment comprises a phoropter; and
the candidate command is a command to perform an adjustment selected from the group consisting of:
adjusting a spherical lens power,
adjusting a cylindrical lens power,
adjusting an axis of a cylindrical lens,
adjusting a position of a Jackson Cross Cylinder lens,
adjusting an add power, and
adjusting an occluded state of a lens.

11. The method of claim 10, wherein:
the client device comprises a phoropter control device;
each of the one or more command-specific executable instructions specifies (i) a command identifier for the adjustment and (ii) a parameter that quantifies a degree of the adjustment;
the output data is formatted for an Application Programming Interface (API) of the phoropter control device; and
wherein receipt of the output data by the phoropter control device causes execution of the one or more command-specific executable instructions to perform the adjustment.

12. The method of claim 1, wherein the change to the configuration comprises updating an eye chart displayed on a graphical user interface of the client device.

13. The method of claim 1, wherein parsing the model output data comprises:
determining a degree of similarity between the model output data and one or more stored responses;
in response to determining that the degree of similarity satisfies a similarity threshold, generating the one or more command-specific executable instructions; and
in response to determining that the degree of similarity does not satisfy the similarity threshold, generating an output inquiry.

14. The method of claim 13, further comprising:
in response to generating the output inquiry, incrementing a counter; and
in response to determining that the counter satisfies a count threshold, establishing a network connection between the client device and a remote provider device.

15. A system comprising:
one or more computing devices; and
one or more storage devices storing instructions that, when executed by the one or more computing devices, causes the one or more computing devices to perform operations comprising:
obtaining, from a plurality of external data sources, context data, wherein the context data comprises (i) historical vision examination data for a patient received from one or more databases, (ii) a current state of a vision examination being administered for the patient;
determining a candidate command for adjusting a hardware component of optometric equipment based on the current state of the vision examination;
generating prompt data for one or more trained machine learning models based on the candidate command, wherein the prompt data specifies a natural language query of the candidate command for a proposed change to a configuration of the hardware component;
providing the prompt data to a hosting system associated with the one or more trained machine learning models;
obtaining, from the hosting system, model output data generated by the one or more trained machine learning models in response to providing the prompt data;
parsing the model output data based on the candidate command to generate one or more command-specific executable instructions for the hardware component; and
providing output data representing the one or more command-specific executable instructions for output, wherein the output data, when received by a client device, causes the client device to initiate a change to a configuration of the hardware component.

16. The system of claim 15, wherein
the context data further comprises input data from the patient received from a remote patient device during the administration of the vision examination; and
the candidate command is determined based on the historical vision examination data and the input data from the patient.

17. The system of claim 16, wherein generating the prompt data comprises structuring the query to integrate the candidate command, the historical vision examination data, the current state of the vision examination, and the input data from the patient.

18. At least one non-transitory computer-readable storage device storing instructions that, when received by one or more processors, causes the one or more processors to perform operations comprising:
obtaining, from a plurality of external data sources, context data, wherein the context data comprises (i) historical vision examination data for a patient received from one or more databases, (ii) a current state of a vision examination being administered for the patient;
determining a candidate command for adjusting a hardware component of optometric equipment based on the current state of the vision examination;
generating prompt data for one or more trained machine learning models based on the candidate command, wherein the prompt data specifies a natural language query of the candidate command for a proposed change to a configuration of the hardware component;
providing the prompt data to a hosting system associated with the one or more trained machine learning models;
obtaining, from the hosting system, model output data generated by the one or more trained machine learning models in response to providing the prompt data;
parsing the model output data based on the candidate command to generate one or more command-specific executable instructions for the hardware component; and
providing output data representing the one or more command-specific executable instructions for output, wherein the output data, when received by a client device, causes the client device to initiate a change to a configuration of the hardware component.

19. The at least one non-transitory computer-readable storage device of claim 18, wherein:
the optometric equipment comprises a phoropter; and
the candidate command is a command to perform an adjustment selected from the group consisting of:
adjusting a spherical lens power,
adjusting a cylindrical lens power,
adjusting an axis of a cylindrical lens,
adjusting a position of a Jackson Cross Cylinder lens,
adjusting an add power, and
adjusting an occluded state of a lens.

20. The at least one non-transitory computer-readable storage device of claim 19, wherein:
the client device comprises a phoropter control device;
each of the one or more command-specific executable instructions specifies (i) a command identifier for the adjustment and (ii) a parameter that quantifies a degree of the adjustment;
the output data is formatted for an Application Programming Interface (API) of the phoropter control device; and
wherein receipt of the output data by the phoropter control device causes execution of the one or more command-specific executable instructions to perform the adjustment.
